(19) United States
(12) Patent Application Publication
(10) Pub. No.: US 2026/0216119 A1
(43) Pub. Date: Jul. 30, 2026

(54) NOVEL SOLUTIONS

(71) Applicant: EDVINCE AB, Lund (SE)

(72) Inventor: Lars EDVINSSON, Lund (SE)

(21) Appl. No.: 19/141,944

(22) PCT Filed: Dec. 20, 2023
(86) PCT No.: PCT/EP2023/087077

§ 371 Date: Jun. 20, 2025

(30) Foreign Application Priority Data
Dec. 21, 2022 (GB) 2219470.8

(51) Int. Cl. A61K 31/277; A61K 9/08; A61K 47/10; A61P 9/10

(52) U.S. Cl. CPC A61K 31/277; A61K 9/08; A61K 47/10; A61P 9/10

(57) ABSTRACT

The invention provides a pharmaceutical composition that is an aqueous solution comprising compound (I) (also known as U0126) or a pharmaceutically-acceptable salt thereof, at a concentration of 50 to 200 μM and wherein the solution further comprises 0.2 to 1.0% (v/v) polyoxyethylene castor oil. The composition finds particular use in the treatment of ischemic damage (in particular subarachnoid hemorrhage) in a human patient by direct injection into the brain. Uses of the composition, unit doses containing the composition and methods of using the composition are also disclosed.

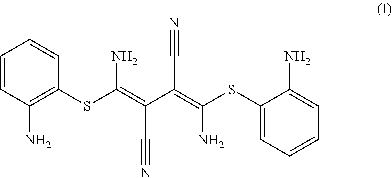 [SMILES: N#CC(=C(/N)Sc1ccccc1N)/C(C#N)=C(\N)Sc1ccccc1N]

### FIELD OF THE INVENTION

The present invention relates to solutions of pharmaceutically-active compounds, in particular for use in the treatment of ischemic damage, especially in the brain. Specifically, the invention relates to an aqueous solution comprising the compound known as U0126 or a pharmaceutically-acceptable solvate of salt thereof, and its use in the treatment of ischemic damage.

### BACKGROUND OF THE INVENTION

Subarachnoid hemorrhage (SAH) is an uncommon form of stroke caused by bleeding in the space that surrounds the brain. Most often, it occurs when a weak area in a blood vessel (aneurysm) on the surface of the brain bursts and leaks. The blood then builds up around the brain and inside the skull, increasing pressure on the brain. SAH is difficult to treat and can be fatal.

One of the main complications of SAH is secondary cerebral ischaemia (also called delayed ischemia), caused by vasospasm—constriction of blood vessels and restriction of blood flow in the brain. Blood supply to parts of the brain can become reduced to a dangerous extent, thereby disrupting the normal functions of the brain.

After confirmation that the SAH was caused by a brain aneurysm, a surgical procedure to repair the affected blood vessel and prevent the aneurysm from bursting again may be recommended. This can be carried out using one of 2 main techniques: coiling and clipping. Clipping involves opening the skull to locate the aneurysm and placing clips around the neck of the aneurysm. Coiling involves advancing a catheter through the arteries that supply the brain via the groin, and depositing platinum coils inside the aneurysm, causing a blood clot to form which limits blood flow.

Secondary cerebral ischemia can be mitigated by pharmacological intervention with the calcium channel blocker nimodipine. The dosing of nimodipine recommended by the UK's National Institute for Health and Care Excellence is 60 mg every 4 hours, to be started within 4 days of aneurysmal subarachnoid haemorrhage, and continued for 21 days.

From preliminary, pre-clinical testing the compound U0126 has shown promise for the treatment of SAH. U0126 has the formula:

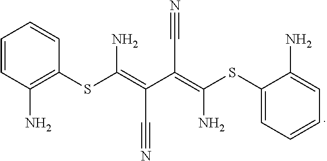 [SMILES: N#CC(=C(/N)Sc1ccccc1N)/C(C#N)=C(\N)Sc1ccccc1N]

It is known to be an inhibitor of MEK1 and MEK2, and has been proposed for use in the treatment of ischemic disorders, for example subarachnoid haemorrhage. Duncia et al., Bioorg. & Med. Chem. Lett. 8 (1998) 2839-2844 describes this activity. EP 1,139,512 discloses the use of U0126 for administration from 1 to 6 hours after onset of ischemic damage. J. Cerebral Blood Flow & Metabolism, 35 (2015) 454-460 discloses that U0126 improves long-term neurologic outcome after stroke in female rats.

U0126 is a difficult compound to formulate and administer, being highly insoluble in most common solvents, and sensitive to both acid and base. Formulations which are soluble in physiological fluids, and which are hence able to transport the active material directly to the brain for the treatment of ischemic disorders, are required for the treatment of ischemic disorders. Intracranial injection is one required mode of administration, and this means that the volume of liquid used as a carrier must be limited. Further, stability of liquid formulations can be an issue. U0126 is known to be soluble in DMSO, but this solvent is not suitable for use in liquid formulations, especially those intended for intracranial injection.

Christensen et al., Drug Delivery, 2019, 26(1), 680-688, describes a composition of U0126 in a 1% solution of Kolliphor EL (also known as Cremophor). Kolliphor EL is a type of polyethoxylated castor oil, prepared by reacting 35 moles of ethylene oxide with each mole of castor oil.

In Christensen et al., ex vivo and in vivo experiments were carried out to determine the optimal dose of U0126 ex vivo and to test the toxicology of this dose in vivo, and to determine synergistic effects between Cremaphor and U0126. The toxicological effect of different concentrations of U0126 in rats was investigated. The animals were tested using three different concentration levels of U0126: low (1×10⁻⁶ M), middle (1×10⁻⁵M) and high (2×10⁻⁵M). 15 μL was injected in each case. It was found in the neurological observations, that the low and middle doses of U0126 were well tolerated. However, at the high level, there were signs of neurological impairment. On that basis, the authors concluded that a concentration of up to 10-5 M U0126 in 0.5% Cremophor (i.e. 10 μM with 0.5% Cremophor in aqueous solution) was the maximum tolerated intracerebroventricular (ICV) administration in vivo dose.

There remains a need for improved treatments for ischemic damage, in particular in the brain.

### SUMMARY OF THE INVENTION

The invention provides a pharmaceutical composition that is an aqueous solution comprising compound (I):

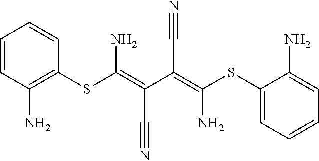 [SMILES: N#CC(=C(/N)Sc1ccccc1N)/C(C#N)=C(\N)Sc1ccccc1N]

or a pharmaceutically-acceptable salt thereof, at a concentration of 50 to 200 μM, wherein the solution further comprises 0.2 to 1.0% (v/v) polyoxyethylene castor oil.

The invention also provides a method for the treatment of ischemic damage, comprising administering the aqueous solution of compound (I) described above to a human patient.

The invention also provides the aqueous solution of compound (I) described above for the manufacture of a medicament for the treatment of ischemic damage.

The invention also provides a unit dose comprising the aqueous solution of compound (I) described above, wherein the unit dose has a volume of 1 to 10 mL.

### DETAILED DESCRIPTION

As discussed above, the invention provides a pharmaceutical composition that is an aqueous solution comprising compound (I):

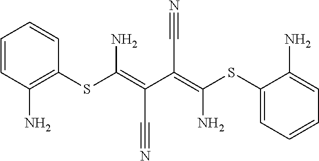 [SMILES: N#CC(=C(/N)Sc1ccccc1N)/C(C#N)=C(\N)Sc1ccccc1N]

or a pharmaceutically-acceptable salt thereof, at a concentration of 15 to 200 μM. The compound of formula (I) is known as U0126. Its chemical name is 1,4-Diamino-2,3-dicyano-1,4-bis(o-aminophenylmercapto) butadiene.

The invention also provides an aqueous solution comprising compound (I) or a pharmaceutically-acceptable salt thereof, at a concentration of 15 to 200 μM.

The U0126 used in the pharmaceutical compositions or solutions of the invention may be in the form of free U0126 or one of its pharmaceutically acceptable salts, for example a salt with a mineral acid or any other acid capable of forming a salt with U0126.

U0126 forms solvates, and these may be used to prepare the compositions or solutions of the invention. Suitable solvates include solvates with alcohols, for example C₁₋₄alkanols, for example ethanol. The hemiethanolate, ethanolate and diethanolate forms all exist and may be used.

The current inventors have surprisingly found that the solution is tolerated in the body at a concentration of 15 to 200 μM and also that it is stable. Based on the earlier reports that 10 μM was the maximum tolerated dose, it was not expected that a concentration as high as this would be tolerated in the body.

In a preferred embodiment, the solution comprises compound (I) at a concentration of about 25 to about 150 μM, for example about 25 to about 100 μM. More preferably, the concentration is about 50 to about 90 μM, for example about 70 to about 80 μM. For example, the solution of the invention comprises compound (I) at a concentration of about 75 μM.

In view of the a molar mass of U0126 of 380.5 g/mol, the solutions comprise compound (I) at a concentration of about 9.5 to about 76.1 μg/mL. More preferably, the concentration is about 9.5 to about 57.1 μg/mL, for example about 9.5 to about 38.5 μg/mL. More preferably, the concentration is about 19.0 to about 34.2 g/mL, for example about 26.6 to about 30.44 μg/mL. For example, the solution of the invention comprises compound (I) at a concentration of about 28.5 μg/mL.

The term ‘about’ refers to a tolerance of ±20% of the relevant value, for example ±15% of the relevant value, such as ±10% of the relevant value or ±5% of the relevant value, for example ±2% of the relevant value.

The pharmaceutical composition of the invention provide the very beneficial advantage that it can be used in a small enough volume in the brain (typically 0.1 to 5 mL, preferably under 2 mL) to not be harmful to the brain, and yet provide sufficient quantity of the drug to be effective.

When administered in a volume of 0.2 to 5 ml, the pharmaceutical composition of the invention will provide a dose to the patient of 0.003 to 1.0 μmol of U0126. As U0126 has a molar mass of 380.5 g/mol, this is a dose of 1.14 to 380.5 μg of U0126. Preferably, the volume and concentration are selected so as to provide a dose in the range 5 to 100 μg of U0126, preferably 5 to 80 μg of U0126, for example 5 to 50 μg of U0126. In a more preferred embodiment, the dose is in the range 10 to 40 μg. Even more preferably, the dose is in the range 20 to 30 μg. For example, when a dose of 0.33 mL of 75 μM U0126 solution is given to a patient, that is a dose of 8.55 μg. When a dose of 1.0 ml of 75 μM U0126 solution is given to a patient, that is a dose of 28.5 μg.

As described below, a course of treatment may involve multiple doses of the drug being given to the patient.

In a preferred embodiment of the invention, the solution further comprises polyoxyethylene castor oil. Polyoxyethylates of castor oil are condensates of ethylene oxide with castor oil, and some are commercially available. Such polyoxyethylene castor oil derivatives may for example be prepared by reacting castor oil with ethylene oxide, for example in a molar ratio of 1:30 to 1:40, for example 1:35. Such products are referred to as Macrogolylycerol ricinoleate, PEG-35 castor oil, Polyoxyl 35 hydrogenated castor oil, and Polyoxyl-35 castor oil. For example, polyoxyl 35 castor oil, for example as available under the trade name Kolliphor® EL, previously known as “Cremophor EL” (Trade Mark).

The pharmaceutical composition or the solution of the invention may comprise the polyoxyethylene castor oil at a concentration of 0.2 to 1.0% (v/v). For example, the solution may comprise the polyoxyethylene castor oil at a concentration of about 0.3 to about 0.7% (v/v), for example about 0.5% (v/v). For example, it may comprises polyoxyl 35 castor oil at a concentration of about 0.3 to about 0.7% (v/v), for example about 0.5% (v/v).

The term “solution” herein is used to describe a homogeneous mixture of the solvent (for example water) and solute (in particular U0126). The mixture passes through a filter (for example a 0.20 μm filter) without any of the composition being held in the filter. In some situations, the solution may, at sufficiently high magnification, be a suspension in which a first solute is surrounded by a second solute and it is the second solute that is in contact with the bulk solvent.

The compound U0126 in solid form remains intact for several decades under ambient conditions. A solution as described herein has been found to remain intact to an extent acceptable for pharmaceutical use for several hours. A solution that remains intact to an extent acceptable for pharmaceutical use for the time required between preparing the solution and administering it to the patient (for example 2 hours, for example 1 hour) is considered herein to be “stable”. It has also been found that the solutions can be frozen and that degradation in the solid state occurs only very slowly.

In certain embodiments, it can be advantageous for the pharmaceutical composition or solution to further comprise a buffer solution.

In certain embodiments, the solution further comprises artificial cerebrospinal fluid (aCSF).

“Artificial cerebrospinal fluid” is a term of art, and aCSF is widely used in research and in medicine, often as an improvement over the use of sterile saline. The composition of aCSF is designed to mirror as closely as possible the composition of human cerebrospinal fluid. Such fluids are sterile aqueous solutions of electrolytes including sodium, potassium, calcium and magnesium cations, together with anions selected from phosphate, sulfate, chloride and bicarbonate. Sugars such as glucose or dextrose may also be included. A typical aCSF comprises, for example, Na⁺: between 130 mM and 170 mM, K⁺: between 2 mM and 5 mM, Ca²⁺: between 1 mM and 2.5 mM, Mg²⁺: between 0.5 mM and 2.5 mM. Suitably, the aCSF comprises Na⁺: between 140 mM and 160 mM, K⁺: between 2.5 mM and 4.5 mM, Ca²⁺: between 1 mM and 2 mM, Mg²⁺: between 0.5 mM and 1.5 mM. More suitably, the aCSF comprises Na⁺: between 145 mM and 155 mM, K⁺: between 2.5 mM and 4 mM, Ca²⁺: between 1 mM and 1.5 mM, Mg²⁺: between 0.5 mM and 1.3 mM. Most suitably, the aCSF comprises Na⁺: about 150 mM, K⁺: between 3 mM and 4 mM, Ca²⁺: between 1.2 mM and 1.5 mM, Mg²⁺: between 0.7 mM and 1.3 mM.

Counterions included in the aCSF may be, for example, phosphate, between 0.3 mM and 1.5 mM, chloride, between 130 mM and 165 mM, and/or bicarbonate, between 20 mM and 30 mM.

In certain embodiments, the aCSF comprises Na⁺: between 140 mM and 160 mM, K⁺: between 2.5 mM and 4.5 mM, Ca²⁺: between 1 mM and 2 mM, Mg²⁺: between 0.5 mM and 1.5 mM, P: between 0.3 mM and 1.5 mM, and Cl⁻: between 130 mM and 165 mM. Suitably, the aCSF comprises Na⁺: between 145 mM and 155 mM, K⁺: between 2.5 mM and 4 mM, Ca²⁺: between 1 mM and 1.5 mM, Mg²⁺: between 0.5 mM and 1.5 mM, P: between 0.4 mM and 1.2 mM, and Cl⁻: between 130 mM and 155 mM. More suitably, the aCSF comprises Na⁺: between 145 mM and 155 mM, K⁺: between 3 mM and 4 mM, Ca²⁺: between 1 mM and 1.5 mM, Mg²⁺: between 0.7 mM and 1.3 mM, P: between 0.4 mM and 1.1 mM, and Cl⁻: between 145 mM and 155 mM.

One suitable aCSF comprises: Na⁺: 150 mM, K⁺: 3 mM, Ca²⁺: 1.4 mM, Mg²⁺: 0.8 mM, P: 1.0 mM, and Cl⁻: 155 mM.

In other embodiments, the aCSF comprises, per 500 ml of artificial cerebrospinal fluid:
between 3500 and 3800 mg sodium chloride;
between 900 and 1000 mg sodium bicarbonate;
between 350 mg and 450 mg dextrose;
between 120 mg and 180 mg magnesium sulfate·7H₂O;
between 120 mg and 180 mg potassium chloride;
between 80 mg and 120 mg calcium chloride·2H₂O;
between 45 mg and 60 mg sodium phosphate, dibasic, anhydrous; and
balance distilled water.

In some embodiments, the aCSF comprises, per 500 ml of artificial cerebrospinal fluid:
between 3600 and 3700 mg sodium chloride;
between 930 and 990 mg sodium bicarbonate;
between 375 mg and 425 mg dextrose;
between 140 mg and 160 mg magnesium sulfate·7H₂O;
between 140 mg and 160 mg potassium chloride;
between 90 mg and 110 mg calcium chloride·2H₂O;
between 50 mg and 55 mg sodium phosphate, dibasic, anhydrous; and
balance distilled water.

In some embodiments, the aCSF comprises, per 500 ml of artificial cerebrospinal fluid: between 3650 and 3690 mg sodium chloride;
between 950 and 970 mg sodium bicarbonate;
between 390 mg and 410 mg dextrose;
between 145 mg and 155 mg magnesium sulfate·7H₂O;
between 145 mg and 155 mg potassium chloride;
between 95 mg and 105 mg calcium chloride·2H₂O;
between 50 mg and 55 mg sodium phosphate, dibasic, anhydrous; and
balance distilled water.

A particularly preferred aCSF consists of, per 500 ml of artificial cerebrospinal fluid:
3670 mg sodium chloride;
960 mg sodium bicarbonate;
400 mg dextrose;
150 mg magnesium sulfate·7H₂O;
150 mg potassium chloride;
100 mg calcium chloride·2H₂O;
53 mg sodium phosphate, dibasic, anhydrous; and
balance distilled water.

In some embodiments, the aCSF consists of, per ml of artificial cerebrospinal fluid:
6.0 to 8.0 mg sodium chloride;
0.2 to 0.4 mg potassium chloride;
0.15 to 0.25 mg calcium chloride·2H₂O;
0.05 to 0.15 mg magnesium chloride hexahydrate;
1.2 to 2.3 mg sodium hydrogen carbonate;
0.05 to 0.15 mg disodium phosphate dihydrate;
Ca 4.5 mg Hydrochloric acid as 1M solution; and
balance distilled water.

A more preferred aCSF consists of, per ml of artificial cerebrospinal fluid:
6.5 to 7.5 mg sodium chloride;
0.25 to 0.35 mg potassium chloride;
0.15 to 0.25 mg calcium chloride·2H₂O;
0.05 to 0.15 mg magnesium chloride hexahydrate;
1.5 to 2.0 mg sodium hydrogen carbonate;
0.05 to 0.15 mg disodium phosphate dihydrate;
Ca 4.5 mg Hydrochloric acid as 1M solution; and
balance distilled water.

For example, the aCSF consists of, per ml of artificial cerebrospinal fluid:
7.2 mg sodium chloride;
0.3 mg potassium chloride;
0.2 mg calcium chloride·2H₂O;
0.1 mg magnesium chloride hexahydrate;
1.8 mg sodium hydrogen carbonate;
0.1 mg disodium phosphate dihydrate;
Ca 4.5 mg Hydrochloric acid as 1M solution; and
balance distilled water.

The invention therefore provides a pharmaceutical composition or a solution that comprises per ml:
9.5 to 76.1 μg U0126;
3.0 to 7.0 mg polyoxyethylene castor oil
6.0 to 8.0 mg sodium chloride;
0.2 to 0.4 mg potassium chloride;
0.15 to 0.25 mg calcium chloride·2H₂O;
0.05 to 0.15 mg magnesium chloride hexahydrate;
1.2 to 2.3 mg sodium hydrogen carbonate;
0.05 to 0.15 mg disodium phosphate dihydrate;
Ca 4.5 mg Hydrochloric acid as 1M solution; and
balance distilled water.

Preferably, the pharmaceutical composition or solution comprises per ml:
12.5 to 50.0 μg U0126;
3.5 to 6.5 mg polyoxyethylene castor oil
6.5 to 7.5 mg sodium chloride;
0.25 to 0.35 mg potassium chloride;
0.15 to 0.25 mg calcium chloride·2H₂O;
0.05 to 0.15 mg magnesium chloride hexahydrate;
1.5 to 2.0 mg sodium hydrogen carbonate;
0.05 to 0.15 mg disodium phosphate dihydrate;
Ca 4.5 mg Hydrochloric acid as 1M solution; and
balance distilled water.

For example, the pharmaceutical composition or solution of the invention consists essentially of the components listed above.

More preferably, the pharmaceutical composition or solution comprises per mL:
19.0 to 38.0 μg U0126;
4.0 to 6.0 mg polyoxyethylene castor oil
6.5 to 7.5 mg sodium chloride;
0.25 to 0.35 mg potassium chloride;
0.175 to 0.225 mg calcium chloride·2H₂O;
0.075 to 0.125 mg magnesium chloride hexahydrate;
1.5 to 2.1 mg sodium hydrogen carbonate;
0.075 to 0.125 mg disodium phosphate dihydrate;
ca 4.5 mg Hydrochloric acid as 1M solution; and
balance distilled water.

For example, the pharmaceutical composition or solution of the invention consists essentially of the components listed above.

For example, the pharmaceutical composition or solution comprises per ml:
28.5 μg U0126;
5.0 mg polyoxyethylene castor oil
6.0 mg sodium chloride;
0.30 mg potassium chloride;
0.20 mg calcium chloride·2H₂O;
0.10 mg magnesium chloride hexahydrate;
1.8 mg sodium hydrogen carbonate;
0.10 mg disodium phosphate dihydrate;
Ca 4.5 mg Hydrochloric acid as 1M solution; and
balance distilled water.

For example, the pharmaceutical composition or solution of the invention consists essentially of the components listed above.

It is preferred for the pharmaceutical composition or solution of the invention to be essentially free of dimethyl sulfoxide (DMSO). However, in some embodiments, the pharmaceutical composition or solution may further comprise dimethyl sulfoxide (DMSO) in a quantity that is low enough to be tolerated in the human body.

The pharmaceutical composition or solution according to the invention may comprise one or more further therapeutic agents. Any desired additional active ingredient may be used. For example a further therapeutic agent may be selected from an anti-inflammatory agent (for example a steroid, e.g. dexamethasone), a calcium channel blocker (for example nimodipine), or an inhibitor of a component of an inflammation response (for example a TNFα or interleukin blocker, typically an antibody).

The solution may be prepared by a process which comprises admixing the ingredients together, followed if necessary by filtration, e.g. microfiltration. The use of vigorous solubilisation techniques may be desirable, for example vigorous stirring or shaking, or the application of ultrasound. The admixing may take place at any suitable temperature and/or pressure. Suitably, the ingredients are mixed together at a temperature of between 20° C. and 45° C., suitably between 25° C. to 40° C. and most suitably between 25° C. and 30° C. Furthermore, the ingredients are preferably mixed together at atmospheric pressure (e.g. 1 atm). As the composition of the invention should be sterile, the process of the invention should be carried out under controlled conditions, for example the process may include sterile filtration with cooling.

The solution may be prepared extemporaneously, or it may be pre-prepared and presented in unit dosage or multi dosage form. It may be presented in kit form, i.e. in the form of two or more separate components, where a component (a) comprising the U0126 is supplied together with a component (b) comprising the polyoxyethylene castor oil and optionally a component (c) the aCSF. Preferably, component (a) is provided as a powder (e.g. as a powder of lyophilised U0126). The powder may then be formed into a solution of the invention by combining with component (b) and component (c). The invention therefore further provides a kit suitable for the preparation of a solution of the invention which comprises as separate components (a) a powder comprising U0126, and (b) a polyoxyethylated castor oil. The kit may optionally also contain (c) pharmaceutically acceptable isotonic solution (e.g. aCSF). The kit may comprise a dry component (such as a powder) comprising U0126, and a liquid component comprising a mixture of polyoxyethylated castor oil and pharmaceutically acceptable isotonic solution (e.g. aCSF). The kit may also comprise a dry component (such as a powder) comprising U0126 and the dry mass ingredients that make up the pharmaceutically acceptable isotonic solution (e.g. aCSF), and a liquid component comprising a polyoxyethylated castor oil.

The mass of the solid components and the volume of the liquid components in a kit of the invention are selected so as provide a pharmaceutical composition or solution of the invention when they are reconstituted. The kit is preferably provided with instructions for the preparation of a pharmaceutical composition or solution of the invention as defined herein.

For example, immediately before use, the dry U0126 is reconstituted using pharmaceutically acceptable isotonic solution (e.g. aCSF) in the presence of the polyoxyethylated castor oil, the aCSF being either supplied with the kit or supplied by the pharmacist or medical professional carrying out the reconstitution. In embodiments in which the kit comprises a powder comprising U0126 and the dry mass ingredients that make up the pharmaceutically acceptable isotonic solution (e.g. aCSF), immediately before use, the powder is reconstituted using (sterile) distilled water, thereby forming the pharmaceutically acceptable isotonic solution in situ (e.g. aCSF). The polyoxyethylated castor oil component of the kit may be added either together with the distilled water or separately therefrom (e.g. before or after the addition of the distilled water).

Suitable powders may be prepared by known methods, for example by lyophilisation. Of course, the composition of the invention should be sterile, and therefore the components supplied as part of the kit should also be sterile.

The invention further provides a pharmaceutically acceptable solution comprising a polyoxyethylated castor oil and artificial cerebrospinal fluid. Such a solution finds particular use in the kit of the invention. For example, such a solution comprises 3.0 to 7.0 mg polyoxyethylene castor oil and an aCSF. For example, such a solution comprises, per mL:
3.0 to 7.0 mg polyoxyethylene castor oil
6.0 to 8.0 mg sodium chloride;
0.2 to 0.4 mg potassium chloride;
0.15 to 0.25 mg calcium chloride·2H₂O;
0.05 to 0.15 mg magnesium chloride hexahydrate;
1.2 to 2.3 mg sodium hydrogen carbonate;
0.05 to 0.15 mg disodium phosphate dihydrate; and
Ca 4.5 mg Hydrochloric acid as 1M solution,
the balance being water.

Based on the findings described herein, the solution of the invention is suitable for use as a medicament. The pharmaceutical compositions and solutions described herein find particular use in the treatment of ischemic disorders, especially subarachnoid haemorrhage.

The invention provides an aqueous solution comprising U0126 or a pharmaceutically acceptable solvate or salt thereof, at a concentration of 15 to 100 μg, for use as a medicament. The preferred and exemplary forms of the solution described hereinabove are also provided for use as a medicament.

Preferably, the solution is for use in the treatment of ischemic damage in a human patient.

Prior to the present invention, U0126 has never been injected intracranially. Accordingly, the invention further provides U0126 or a pharmaceutically acceptable salt or solvate thereof for use in the treatment of an ischemic disorder, said treatment being by direct injection of a solution of U0126 or a pharmaceutically acceptable salt or solvate thereof into the brain; and a method for treating an ischemic disorder which comprises injecting a solution comprising U0126 or a pharmaceutically acceptable or solvate salt thereof directly into the brain.

The invention also provides a method of treating ischemic damage in a human patient comprising administration of a therapeutically effective amount of a solution of the invention. Preferably, the composition is administered by injection. In an especially preferred embodiment, the composition is administered by intracerebroventricular (ICV) injection.

The invention also provides the use of the solution of the invention for the manufacture of a medicament the treatment of an ischemic disorder. Preferably, the ischemic disorder is subarachnoid haemorrhage.

The solution of the invention may be provided in unit dose form. In certain embodiments, the invention provides a unit comprising a volume of 0.2 to 10 ml of the solution from which a treatment dose can be taken. In a preferred embodiment, the unit has a volume of 3 to 9 mL, for example 5 to 8 mL, for example 7 mL. Typically, a dosing volume administered to a patient is in the range 0.1 to 5 mL. Preferably it is under 2 mL, for example it may be 0.2 to 1.5 mL, preferably 0.3 to 1.0 mL, for example 0.33 mL or 1.0 mL.

### EXAMPLES

### Example 1: Preparation of a Solution of U0126 in 0.5% Cremophor EL in aCSF (Tocris Pharma)

A solution of sodium chloride, potassium chloride, calcium chloride dihydrate, and magnesium chloride hexahydrate was prepared by mixing in water (Water For Injection grade). The pH was adjusted to 2.8 using 1M HCl and/or 0.5M NaOH. A second solution was prepared by mixing sodium hydrogen carbonate and disodium phosphate dihydrate in water (Water For Injection grade). The first and second solutions were mixed together to form an aCSF solution.

U0126 drug product was dissolved in an aqueous solution of Kolliphor EL. That was then added to the aCSF solution. The solution was prepared in three 1 litre batches and combined to a provide a final volume of 3 L.

The solution was sterile filtered using 2 Millipak 100 0.22 μm PVDF filter units. The solution was then filled into vials and they were sealed. The products were checked for purity and correct final concentrations. The final concentrations of the components were as shown in Table 1:

TABLE 1
Concentrations of components of solution
of U0126 in 0.5% Cremophor EL in aCSF

| Component | Amount/mL | |
|---|---|---|
| U0126 | 28.5 | μg |
| Kolliphor EL | 5.0 | mg |
| Sodium chloride | 7.2 | mg |
| Potassium chloride | 0.3 | mg |
| Calcium chloride dihydrate | 0.2 | mg |
| Magnesium chloride hexahydrate | 0.1 | mg |
| Sodium hydrogen carbonate | 1.8 | mg |
| Disodium phosphate dihydrate | 0.1 | mg |

| | |
|---|---|
| Hydrochloric Acid, as 1M solution | q.s. (ca. 4.5 mg) |
| Water | To 1 ml |

That is to say that the solution contained 28.55 μg/ml of U0126 (75 μM) and 0.5% v/v of Kolliphor EL.

### Example 2: Alternative Preparation of a Solution of the Invention

0.79 g Cremophor EL (Trade Mark) was weighed in an Erlenmeyer flask, and 60 mL aCSF (obtained from Tocris Pharma) was added. The mixture was shaken vigorously for 15 minutes, resulting in a clear solution containing 1.2% v/v of Cremophor EL. 4.88 mg of U0126 was added to the solution, together with an additional 15 mL aCSF. These quantities were calculated to give 194 μM U0126 in a 1.0% v/v solution of Cremophor EL in aCSF. The solution was then placed in an ultrasonic bath maintained at 25-28° C. for 20 minutes. After ultrasonication, some particles were observed in the solution. The solution was ultrasonicated for another 25 minutes. The resulting mixture was diluted with an additional 74.25 mL aCSF solution to give a calculated 78.75 M U0126 in 0.5% v/v solution of Cremophor EL in aCSF. The actual measured content of U0126 was 67 M (HPLC, Agilent 1100).

The mixture was then filtered using a Millipore Millex-LG, PTFE, 0.20 μm filter (SLLG025SS). The filter removed all solid material leaving a clear solution, containing a measured content of U0126 of 68 μM.

### Example 3: Alternative Preparation of a Solution of the Invention

500 μL Cremophor EL was pipetted into an Erlenmeyer flask containing 40 mL in-house prepared aCSF. The mixture was shaken vigorously for 15 minutes resulting in a clear solution containing 1.25% Cremophor EL. To this solution was added 4.21 mg of U0126 and an additional 10 mL aCSF. The mixture was shaken vigorously for 43 minutes resulting in a non-clear solution. An additional 87 minutes of shaking did not dissolve the remaining particles. The mixture was then ultrasonicated for 5 minutes, whereupon the particles dissolved. An additional 10 minutes of ultrasonication was carried out to ensure complete dissolution. The resulting solution was calculated to contain 1.0% Cremophor EL and 200 μM U0126.

This solution was then diluted with 50 mL aCSF to give a solution which was calculated to contain 0.5% Cremophor EL and 98.7 μM U0126. Various volumes of this solution, which had a measured pH of 8.6, were filtered using a Millipore Sterile Syringe Filter, Millex-LG PTFE 0.20 μm (SLLG013SL). Analysis of the various samples showed a U0126 content of from 85-99 μM.

### Biological Example 1: Trial in Human Subjects with SAH

A randomised, double-blind, placebo-controlled trial is carried out to determine the safety and tolerability of the drug product of Example 1 (Investigational Medicinal Product—IMP) administered in 1 and 3 ICV bolus injections over 18 hours to patients in the early phase after subarachnoid haemorrhage (SAH). The trial includes five cohorts of patients and they are administered the drug product of Example 1 (Investigational Medicinal Product—IMP) as follows:
Cohort 1: three patients, low dose IMP, no placebo, single dose
Cohort 2: four patients, medium dose, randomised 3:1 to IMP:placebo, single dose
Cohort 3: four patients, full dose, randomised 3:1 to IMP:placebo, single dose
Cohort 4: four patients, full dose, randomised 3:1 to IMP:placebo, multiple dose
Cohort 5:12 patients, full dose, randomised 3:1 to IMP:placebo, multiple dose

The Investigational Medicinal Product (IMP) is the solution as described above in Example 1. It contains the active pharmaceutical ingredient U0126 at a concentration of 28.5 μg/mL. For the low dose patients, 0.33 mL of the solution is administered. For the medium dose patients, 0.6 mL of the solution is administered For the high dose patients, 1.0 ml of the solution is administered. For the multiple dose patients, three doses of 1.0 ml per dose are administered.

The placebo is 0.9% saline.

The trial has the following inclusion and exclusion criteria:

### Inclusion Criteria:

Patients meeting the following criteria are eligible for participation:
1. Male or female patients aged 18-80 years (both inclusive).
2. Moderate or severe SAH diagnosed with CT and caused (or suspected to be caused) by a ruptured saccular aneurysm with symptoms less than 8 hours.
3. Intracerebroventricular access obtained within 8 h from start of symptoms.
4. A WFNS score 1-5, assessed at any time after SAH diagnosis, but before first dose of IMP.
5. Informed consent obtained from a trial guardian prior to initiation of any trial-related procedures.

### Exclusion Criteria:

Patients are excluded from participation in the trial if any of the following criteria applies:
1. The SAH due to other causes (e.g., trauma, rupture of fusiform or mycotic aneurysms).
2. Intraventricular or intracerebral blood, in the absence of subarachnoid blood.
3. Expected survival <48 hours.
4. Any severe or unstable chronic or acute concomitant condition, which, in the opinion of the PI/delegate, would affect the assessment of the safety of the IMP.
5. Any known or CT evidence of previous major cerebral damage or pre-existing cerebrovascular disorders, which in the opinion of the PI/delegate may affect the accurate diagnosis and evaluation of SAH.
6. Participation in any other clinical trial with an experimental drug within the last 12 weeks.
7. Known allergy to the IMP or its constituents.
8. Female patients who are pregnant or breastfeeding. Women of childbearing potential must have a negative plasma or urine pregnancy test at screening.

### Investigational Medicinal Product (IMP) Preparation and Administration:

The IMP will be prepared at the trial site by an un-blinded pharmacist as detailed in the Pharmacy.

Administration of the IMP or placebo is by intracerebroventricular injection. It is carried out in accordance with the following schedule shown in Table 2:

TABLE 2
Schedule of administration of the IMP or placebo

| | | | Third dose 12 h |
|---|---|---|---|
| | First dose within | Second dose | after the second |
| | 8 h from start of | 6 h after the | dose (end of |
| IMP or Placebo | SAH symptoms | first dose | treatment) |
| Cohort 1, 2 and 3: | X | | |
| Day 1 (0-24 h) | | | |
| Cohort 4 and 5: | X | X* | X* |
| Day 1 (0-24 h) | | | |

*These doses may be on day 2.

After the administration of 0.33 mL, 0.6 mL or 1.0 ml of IMP, an injection of 2-5 ml of isotonic NaCl is also administered. This is to ensure that the entire IMP dose reaches the target. The ICV connection should be kept closed for 15-45 minutes after the injection. During the time the connection is closed, intracranial pressure (ICP) is monitored frequently, to avoid the ICP becoming too high, in the opinion of the Primary Investigator or their Delegate.

For each patient, the following information are recorded as part of the trial protocol:
Demographics (Age, sex, height, weight)
Medical History and SAH Diagnosis
Concomitant medication
Vital signs.
Electrocardiogram: continuous monitoring via a 3 lead ECG throughout the stay in the NICU as per clinical practise.
Magnetic resonance imaging scan: An MRI scan will be performed at the Day 84 visit for documentation of cerebral infarction.
World Federation of Neurosurgical Societies Score: The level of consciousness and neurological deficits will be measured by WFNS score at screening as one of the inclusion criteria and as baseline status before the first IMP administration.
Glasgow Coma Scale (GCS) The GCS is used for assessing the level of consciousness while the patient is hospitalized. The frequency of administration is at least once daily.
Extended Glasgow Outcome Scale The GOS-E is used to evaluate the patient's health status in eight categories for clinical function for health. The eight categories are: dead, vegetative state, lower severe disability, upper severe disability, lower moderate disability, upper moderate disability, lower good recovery, and upper good recovery.
Modified Rankin Scale The mRS will be used to measure the degree of disability or dependence on caregivers in the daily activities.
NIHSS: The NIHSS grading for focal neurological deficits will be evaluated at baseline and as a measurement of disease progression up to 14 days after SAH.
Laboratory safety assessments: Blood samples for analysis of clinical chemistry and haematology are collected. Arterial blood gas parameters are collected and sent to the certified clinical chemistry laboratory at the local laboratory and analysed by routine analytical methods. Blood samples for laboratory safety assessments are collected before each IMP administration (within 1 hour for the second and third dose), 75 minutes (±15 minutes) after each IMP administration and then once daily (except blood gases, unless clinically indicated) during the remaining intensive care period for the patient at NICU.
Pregnancy test
Delayed cerebral ischemia
Registration of length of stay in NICU, in hospital units or other hospitals
Any Exploratory Procedures and Assessments that are carried out.

At the time of filing this patent application, cohorts 1 to 3 are complete and cohort 4 is partly complete. No Serious Adverse Events (SAEs) with a causal relationship to Investigational Medicinal Product preparation or the administration procedure have been reported (including in cohort 4 in which patients receive multiple doses: three doses of 1.0 mL per dose, as set out in the protocol above). The readout of efficacy data will be available once further stages of the study have been completed.

### Biological Example 2: Repeat Dose Toxicity Studies in Dogs

The objective of this study was to assess the toxicity, with emphasis on effects in the brain, of drug product of Example 1 (Investigational Medicinal Product—IMP) when administered once daily for eight days (Days 0-7) by ICV injection in Beagle dogs. In addition, cardiovascular and CNS safety pharmacology assessments were included as an integral part in this study.

The study consisted of five groups of up to five Beagle dogs (up to 3 males and 2 females, see Table 3). A subset of the animals was subjected to magnetic resonance imaging to facilitate location of the cerebroventricular spaces. After imaging and analysis of this subset, each animal was surgically implanted with an ICV cannula in one of the lateral ventricles. Following surgical recovery, Groups 1-5 were dosed via the ICV cannula with (respectively) vehicle, and drug product of Example 1 (Investigational Medicinal Product—IMP) at three different concentrations in a dose volume of 500 μL per dose (followed by 50 μl artificial cerebrospinal fluid). The dose level and number of animals to complete the study are shown in Table 3 below:

TABLE 3
Dose levels and number of animals to complete the study

| | | Number of animals completing the study | |
|---|---|---|---|
| Group | Dose Level (μg) | M | F |
| Control | — | 2 | 2 |
| Low Dose | 3.8 | 3 | 2 |
| Mid Dose | 19 | 2 | 2 |
| High Dose | 38 | 3 | 2 |
| aCSF | — | 1 | 2 |

The administration was by slow ICV injection (~3 μL/sec) once daily over eight days, for a total of (up to) 8 doses. Blood for plasma and serum was collected post-dose on Days 0 and 7. During the treatment period (Days 0-7), the animals were monitored for clinical signs for the first hour post-dose, and up to four times daily. Body weight and food consumption was monitored and functional observation battery assessments were performed prior to start of dosing, and post-dose on Days 0 and 7. Cardiovascular/respiratory examinations were performed post-dose on the first day of dosing.

At sacrifice, animals were subjected to complete gross necropsies, including collection, weighing, fixation and histopathological examination of a full list of organs. The histological evaluation of the brain comprised multiple sections including the injection site (i.e., the injected lateral ventricle), and the contralateral (un-injected) lateral ventricle.

Formulations were made fresh every four days and kept refrigerated until use in accordance with the results of stability studies.

There was no unscheduled mortality. Body weights fell by 5-15% over the course of the treatment duration in all groups, including controls, and there were no significant differences in body weight among the groups. Inappetence or reduced appetence was observed in the majority of animals on one or more days following the start of dosing. Reduced appetence did not appear to correlate with the number of doses or the dose level.

Starting two hours after the first dose administration, dogs in all dose groups exhibited some, or all, of the following signs on one or several occasions throughout the study: passivity, rise in body temperature (values exceeding 101° F./38.3° C.), ear twitching, body/head/jaw shaking, changes in body posture and vocalizing.

Cardiovascular and respiratory function was assessed in conscious, restrained animals approximately 2 h after the first dose. Examinations included 12-lead electrocardiography (ECG), blood pressure, respiratory rate, haemoglobin pulse oximetry, capillary refill time via blood pressure cuff and manual auscultation of the thorax to assess heart beat and lung sounds.

All animals were included in the analysis. There were no treatment-related effects on cardiovascular or respiratory parameters at any dose level.

Extensive and detailed histological evaluation was performed on multiple levels of brain from all animals. Histopathological findings were as follows: inflammatory changes in the ventricles, adjacent neuropil and meninges were present in the brains of all animals at the levels of the injection site and usually to a lesser extent in other areas of the cortex, cerebellum, and brain stem. These findings were also present in the brains of the dogs administered aCSF, and were similar in severity and extent to those seen in vehicle (0.5% Kolliphor EL)-treated animals and in animals receiving any of the three dose levels of test article. Focal haemorrhages, varying in size were generally seen in most dogs in all groups receiving drug product of Example 1 (Investigational Medicinal Product—IMP). Although absent in vehicle-treated animals (but present in 1 of 3 aCSF treated animals), the localisation and the lack of correlation to dose level suggest that these haemorrhages were not the result of test article toxicity, but were due to local mechanical trauma from the catheter tips. There did not appear to be any differences in the incidence or severity of the inflammatory response and haemorrhages related to the number of infusions that each dog received. Apart from the findings in the brain, there were no histopathological findings in any other organ.

Whilst some adverse effects and histopathologic lesions were observed in this study, they were considered to relate to the surgical and infusion procedures rather than direct vehicle- or test article toxicity.

The concentrations of U0126 in the dose formulations used in this toxicity study were higher than in the clinical formulation, except the low dose group in dogs (see Table 4 below). Assuming instantaneous distribution of the drug into the ventricles and the CSF, the Cₘₐₓ of the U0126 achieved in these compartments at the lowest dose in dogs was estimated to be the same as that in humans at the clinical dose of 28.5 μg. In mid- and high dose animals, the estimated Cₘₐₓ in dogs 3.6 and 7.3-fold higher (Table 3).

TABLE 4
Calculated concentrations of U0126 and Kolliphor
EL based on ventricle and CSF-volumes in dog

| | | Volumes | Cₘₐₓ | Cₘₐₓ | Kolliphor |
|---|---|---|---|---|---|
| | Dose | Ventricle/CSF | ventricle | CSF | EL in CSF |
| Dog | aCSF | 2 ml | 0 | 0 | 0 |
| | Kolliphor | 20 mL | 0 | 0 | 0.0125% |
| | EL/0.5 mL | | | | |
| | 20 μM/0.5 mL | | 5 μM | 0.5 μM | 0.0125% |
| | 100 μM/0.5 mL | | 25 μM | 2.5 μM | 0.0125% |
| | 200 μM/0.5 mL | | 50 μM | 5 μM | 0.0125% |

As discussed above, no difference in the local tolerability/toxicity of the U0126 drug product could be identified in the pivotal toxicity studies. The estimated Cₘₐₓ levels (in ventricles and CSF) in the dog were up to 7.3-fold higher than those estimated in human patients at the clinical dosing level.

Hence, the concentration and dose of the drug product of Example 1 (0.33 or 1.0 ml of 75 μM solution) was considered safe for use in human patients.

1. A pharmaceutical composition that is an aqueous solution comprising compound (I) 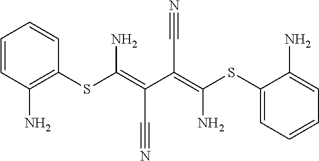 [SMILES: N#CC(=C(/N)Sc1ccccc1N)/C(C#N)=C(\N)Sc1ccccc1N]
or a pharmaceutically-acceptable salt thereof, at a concentration of 50 to 200 μM, wherein the solution further comprises 0.2 to 1.0% (v/v) polyoxyethylene castor oil.

2. The pharmaceutical composition as claimed in claim 1, wherein the concentration of compound (I) in the solution is 50 to 90 μM.

3. The pharmaceutical composition as claimed in claim 2, wherein the concentration of compound (I) in the solution is 75 μM.

4. The pharmaceutical composition as claimed in claim 1, wherein the polyoxyethylene castor oil is polyoxyl 35 castor oil.

5. The pharmaceutical composition as claimed in claim 1, wherein the solution comprises 0.5% (v/v) polyoxyl 35 castor oil.

6. The pharmaceutical composition as claimed in claim 1, wherein the solution further comprises a buffer solution.

7. The pharmaceutical composition as claimed in claim 1, wherein the solution further comprises artificial cerebrospinal fluid (aCSF).

8. The pharmaceutical composition as claimed in claim 1, wherein the solution further comprises dimethyl sulfoxide (DMSO).

9. The pharmaceutical composition as claimed in claim 1, wherein the solution comprises one or more further therapeutic agents.

10. An aqueous solution comprising compound (I) 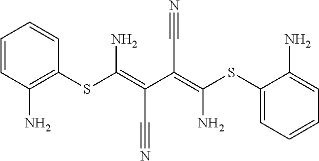 [SMILES: N#CC(=C(/N)Sc1ccccc1N)/C(C#N)=C(\N)Sc1ccccc1N]
or a pharmaceutically-acceptable salt thereof, at a concentration of 50 to 200 μM, wherein the solution further comprises 0.2 to 1.0% (v/v) polyoxyethylene castor oil.

11-13. (canceled)

14. A method for the treatment of ischemic damage comprising administering compound (I) 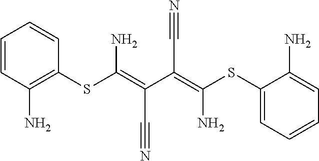 [SMILES: N#CC(=C(/N)Sc1ccccc1N)/C(C#N)=C(\N)Sc1ccccc1N]
or a pharmaceutically-acceptable solvate of salt thereof to a human patient wherein ischemic damage is treated in the patient.

15. A method of manufacture of a medicament for treatment of ischemic damage comprising obtaining compound (I) 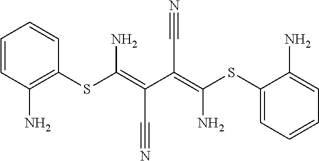 [SMILES: N#CC(=C(/N)Sc1ccccc1N)/C(C#N)=C(\N)Sc1ccccc1N]
or salt thereof and formulating it into an aqueous solution.

16. A unit dose comprising compound (I) 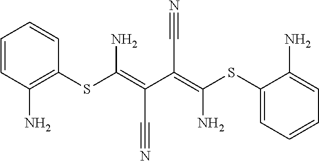 [SMILES: N#CC(=C(/N)Sc1ccccc1N)/C(C#N)=C(\N)Sc1ccccc1N]
or a pharmaceutically-acceptable solvate of salt thereof wherein the unit dose has a volume of 1 to 10 mL.

17. A pharmaceutically acceptable solution comprising a polyoxyethylated castor oil and artificial cerebrospinal fluid.

18. A method of using Compound (I) to treat an ischemic disorder in a human comprising:
obtaining Compound (I): 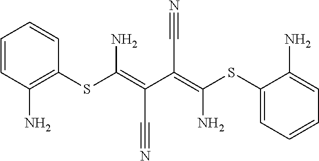 [SMILES: N#CC(=C(/N)Sc1ccccc1N)/C(C#N)=C(\N)Sc1ccccc1N]
or a pharmaceutically-acceptable solvate of salt thereof that has been formulated into a solution appropriate for direct injection into the brain of a human; and
injecting the solution directly into the brain of a human;
wherein an ischemic disorder is treated in the human.

19. The pharmaceutical composition as claimed in claim 2, wherein the concentration of compound (I) in the solution is 70 to 80 μM.

20. The pharmaceutical composition as claimed in claim 4, wherein the polyoxyethylene castor oil is available under the trade name Kolliphor® EL.

21. The unit dose of claim 16, wherein the unit dose has a volume of 7 mL.
